(19) United States
(12) Patent Application Publication
(10) Pub. No.: US 2017/0107254 A1
(43) Pub. Date: Apr. 20, 2017

(54) Modified Antimicrobial Peptides

(71) Applicant: NovaBiotics Limited, Aberdeen (GB)

(72) Inventors: Deborah O'NEIL, Aberdeen (GB); Derry MERCER, Aberdeen (GB); Vanessa DUNCAN, Aberdeen (GB)

(21) Appl. No.: 15/301,472

(22) PCT Filed: Apr. 2, 2015
(86) PCT No.: PCT/GB2015/051039

§ 371 Date: Oct. 3, 2016

Related U.S. Application Data
(60) Provisional application No. 61/974,087, filed on Apr. 2, 2014.

(30) Foreign Application Priority Data
Apr. 2, 2014 (GB) 1405891.1

(51) Int. Cl. C07K 7/08

(52) U.S. Cl. CPC C07K 7/08; A61K 38/00

(57) ABSTRACT

The present invention relates to modified antimicrobial peptides which incorporate a histidine tag, are conjugated with a fatty acid and/or are PEGylated and their use in the treatment of microbial infections.

### FIELD OF THE INVENTION

The present invention relates to modified antimicrobial peptides, formulations comprising such peptides and their use in the treatment of microbial infections, such as those caused by yeasts and/or moulds.

### BACKGROUND TO THE INVENTION

The frequency of invasive fungal infections has continued to increase over the past two decades, both in the general population and in immunosuppressed patients with the vast majority of infections caused by Aspergillus and Candida species (Pasqualotto, A. C., and Denning, D. W. (2005) Diagnosis of Invasive Fungal Infections—Current Limitations of Classical and New Diagnostic Methods. Euro Oncol Rev). These infections carry high mortality rates and place significant burdens on health care systems. There remains an urgent need for more effective and safe therapeutic agents to treat and prevent infections by yeasts and mould (including e.g. Candida spp. or Aspergillus spp.).

Antimicrobial peptides (AMPs) are a diverse group of evolutionary conserved molecules of the innate immune system of most known organisms. These effector molecules are the first line of defence against invading organisms, so are effective against bacteria, fungi, viruses and parasites. Cell membranes of microorganisms tend to be the target for AMPs where they swiftly and physically penetrate causing membrane lysis and death, a mode of action that substantially reduces the risk of acquisition of resistance. Structurally, most endogenous AMPs carry a net positive charge and have a total of 12-50 amino acids with ˜50% of the amino acids being hydrophobic. Small, cationic antimicrobial peptides have also been isolated from many bacteria, fungi, plants, invertebrates and vertebrates and would therefore appear also to play a role in prokaryotic defences.

Natural AMP exhibit broad-spectrum activity against Gram-positive and Gram-negative bacteria, yeasts, fungi and enveloped viruses. Microbial pathogens do not seem to acquire resistance to these cationic peptides and as such, AMP have been conserved as a vital innate immune host defence molecules through millennia of evolution. It is not surprising therefore that AMP have been implicated as potential targets for therapeutics for a wide range of infections. However, the fact that they are technically challenging and costly to produce in recombinant systems and have potent chemotactic and inflammatory biological functions rules out natural AMP forms for as therapeutics.

We have shown that synthetic linear peptides, engineered on the template of the amino acid sequences of the endogenous peptidic structures referenced above, rich in certain basic residues such as lysine or arginine possess antimicrobial activity, and, in particular, antifungal activity. These novel peptides and their lipidated variants may provide one solution to the unmet need for better antifungal agents. Further, we have shown that specific modifications to these peptides, such as the addition of histidine terminal tags and PEGylation can potentially provide improved potency in clinical use.

### STATEMENTS OF THE INVENTION

According to a first aspect of the invention, there is provided a modified peptide comprising from 3 to 50 D and/or L amino acids wherein the amino acids are predominantly arginine and wherein the peptide comprises a modification which is selected from one or more of the group consisting of:
1) Incorporation of a histidine tag;
2) lipidation; and
3) pegylation

The present invention is predicated on the surprising finding that such modified peptides have particular utility in the treatment of microbial infections, suitably fungal infections such as Aspergillus and/or Candida infections for example.

Suitably, the histidine tag may comprise at least two histidine residues.

In an additional or alternative aspect, the modified peptide of the present invention may be a lipidated peptide such that a fatty acid is conjugated to the peptide.

Suitably, the fatty acid may be a C₂ to C₂₀ fatty acid. Preferably, the fatty acid may be C₃ to C₁₄.

In another additional or alternative aspect, the modified peptide of the present invention may be PEGylated.

In another aspect of the present invention there is provided a method of preparing a modified peptide in accordance with the present invention, said method comprising:
1) providing a peptide comprising from 3 to 50 D and/or L arginine amino acids except for to 0, 1 or 2 substitutions; and
2) incorporating a histidine tag; conjugating said peptide with a fatty acid and/or PEGylating said peptide so as to produce a modified peptide.

Modified peptides produced in accordance with the present invention are also encompassed by the present invention.

In a further aspect, the present invention provides a pharmaceutical composition comprising a modified peptide in accordance with any one the preceding claims and a pharmaceutically acceptable carrier, excipient or diluent.

Suitably, the pharmaceutically acceptable composition of the present invention may further comprises a pH stabilising agent.

In another aspect, the present invention provides modified peptides and/or pharmaceutical composition of the present invention are provided for use a medicament.

In a further aspect, the present invention provides modified peptides of the present invention or a pharmaceutical composition of the present invention for use in the prevention or treatment of a microbial infection.

Suitably, the microbial infection may be a fungal infection e.g., a Candida infection and/or an Aspergillus infection. Suitably, the microbial infection may be caused by yeast and/or moulds. Suitably, the microbial infection may an infection by one or more of the group consisting of: Candida spp., (e.g. C. albicans), Epidermophyton spp., Exophiala spp., Microsporum spp., Trichophyton spp., (e.g T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp. (e.g Malassezia furfur), Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

In a further aspect, the present invention provides modified peptides of the present invention or a pharmaceutical composition of the present invention for use in the prevention or treatment of any one or more of the group consisting of: candidiasis (including OPC), aspergillosis (including bronchopulmonary aspergillosis, chronic pulmonary aspergillosis and aspergillomata), athlete's foot; basidiodiabolomycosis; blastomycosis; coccidioidomycosis cryptoccocis; basal meningitis; dermatophytosis; onchomycosis; dermatophytids; endothrix; exothrix; fungal meningitis, fungemia, histoplasmosis, mycosis, myrinogmycosis, paracoccidioidomycosis, penicilliosis, piedra, pneumocytosis pneumonia, sporptrichosis, tinea, zeospora and zygomycosis.

In another aspect, the present invention provides a method of treating or preventing a microbial infection in a subject comprising administering a pharmaceutically effective amount of the modified peptide of the present invention or a pharmaceutical composition of the present invention. Suitably, the microbial infection may an infection by one or more of the group consisting of: Candida spp., (e.g. C. albicans), Epidermophyton spp., Exophiala spp., Microsporum spp., Trichophyton spp., (e.g T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp. (e.g Malassezia furfur), Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp. Suitably, the administration route may be intravenous, infusion, oral, topical or inhaled.

In another aspect, the present invention provides a method of treating or preventing any one or more of the group consisting of: candidiasis (including OPC), aspergillosis (including bronchopulmonary aspergillosis, chronic pulmonary aspergillosis and aspergillomata), athlete's foot; basidiodiabolomycosis; blastomycosis; coccidioidomycosis cryptoccocis; basal meningitis; dermatophytosis; onchomycosis; dermatophytids; endothrix; exothrix; fungal meningitis, fungemia, histoplasmosis, mycosis, myrinogmycosis, paracoccidioidomycosis, penicilliosis, piedra, pneumocytosis pneumonia, sporptrichosis, tinea, zeospora and zygomycosis in a subject, said method comprising administering a pharmaceutically effective amount of the modified peptide of the present invention or a pharmaceutical composition of the present invention. Suitably, the microbial infection may be a fungal infection e.g., a Candida infection and/or an Aspergillus infection. Suitably, the administration route may be intravenous, infusion, oral, topical or inhaled.

Suitably in the methods of the present invention the subject may have HIV or AIDS.

### DETAILED DESCRIPTION

The present invention relates to modified peptide comprising from 3 to 50 D and/or L amino acids wherein the amino acids are predominantly arginine and wherein the peptide comprises a modification which is selected from one or more of the group consisting of:
1) Incorporation of a histidine tag;
2) lipidation; and
3) pegylation

### Incorporation of a Histidine Tag

In one aspect, the modified peptide of the present invention preferably comprises a histidine tag at either the N terminus or C terminus. Advantageously, the presence of a histidine may enhance the effectiveness of the peptide against fungal infections such as Candida. This is extremely unexpected given that the cationic charge may not be significantly changed at such a pH range when compared to an equivalent peptide without the presence of a histidine tag.

Suitably, the histidine tag may comprise at least two histidine residues. Preferably, the number of histidine residues may be up to 10. For example the histidine tag may consist of 1 to 10 histidine residues, preferably 2 to 6. In one embodiment, the histidine tag may consist of two histidine residues

Advantageously, the presence of a histidine tag may be particularly useful for treating fungal infections of the mouth such as oropharyngeal candidiasis.

The oral cavity has a pH between 5.5 and 7 in disease states whereas the normal pH of the mouth of a healthy oral cavity is around pH 7 when not feeding. However, pH influences the charge of AMPs. Furthermore, secreted saliva also contains proteases that aid the breakdown of peptides.

The present inventors have surprisingly found that peptide of the invention modified to comprise a histidine tag are particularly adept at overcoming the pH and protease challenges associated which oral administration.

Accordingly, modified peptides of the present invention comprising a histidine tag may be comprised in pharmaceutical formulations adapted for oral administration.

Suitably, the peptide used in the pharmaceutical compositions of the present invention, method of treatment or prevention of the present invention and second medical uses of the present invention may comprises a histidine tag when the route of administration or intended route of administration is oral administration.

Preferably, the pH of the pharmaceutical compositions of the present invention is in the region of pH 5.5 to 6.5.

### Lipidation

In one aspect, the modified peptides of the present invention are lipidated. For example, a lipid may be conjugated to a peptide comprising from 3 to 50 D and/or L amino acids wherein the amino acids are predominantly arginine.

The present invention has surprisingly found that lipidation of the peptides can advantageously broaden the spectrum of activity of the peptides against microbes and/or enhance the activity of the peptides against some microbial infections.

Suitably, lipidated peptides of the present invention may be used in the treatment or prevention of yeast and mould infections (preferably such as Candida and/or Aspergillus infections, preferably Aspergillus infections). It has been surprisingly found that lipidation of the peptides claimed can confer potent activity on such lipidated peptides.

Accordingly, the modified peptides of the present invention may comprise a lipid which may be at either the C terminus, N terminus or flanked with amino acid residues.

Suitably the peptides of the present invention may comprise a C₃ to C₂₀ fatty acid, preferably a C₄ to C₁₄ fatty acid, preferably a C₈ to C₁₄ fatty acid, preferably a C₁₂ fatty acid.

Suitably the modified peptides of the present invention may comprise 3 to 50 amino acids and a C₃ to C₂₀ fatty acid, preferably a C₄ to C₁₄ fatty acid, preferably a C₈ to C₁₄ fatty acid, preferably a C₁₂ fatty acid. Preferably, the modified peptides of the present invention may comprise 6 to 50 amino acids and a C₃ to C₂₀ fatty acid, preferably a C₄ to C₁₄ fatty acid, preferably a C₈ to C₁₄ fatty acid, preferably a C₁₂ fatty acid.

In one aspect, the fatty acid may be flanked on either side by amino acid residues. It has surprisingly been found that the flanking of the fatty acid can lead to a reduction in haemolytic activity.

In another aspect, the fatty acid may be located on the terminus of the peptide. It has surprisingly been found that this may increase the antimicrobial effects of the peptide in terms of lower MIC.

In one preferable embodiment the fatty acid is a C₁₂ fatty acid. Advantageously, this length of fatty acids exhibits both good antimicrobial effects and additionally has low cytotoxicity and haemolytic activity.

### PEGylated Peptides

In one aspect the modified peptide of the present invention is a PEGylated peptide.

Advantageously, such PEGylated peptides have enhanced stability whilst still providing antimicrobial effects.

Suitably, the size of the PEG component may be approx. 300 Da to approx. 40 KDa.

### Amino Acid Residues

The peptide may comprise from 3 to 50 (preferably contiguous) amino acids.

Suitably the peptide may comprise at least 3 or at least 4 or at least 5 or at least 6 or at least 7 or at least 8 or at least 9 or at least 10 or at least 12 or at least 15 or at least 20 or at least 25 or at least 30 or at least 35 or at least 40 or at least 45 amino acids.

Suitably, the peptide may comprise less than 50 or less than 45 or less than 40 or less than 35 or less than 30 or less than 25 or less than 0 or less than 15 amino acids.

In one aspect the number of amino acid residues referred to in the ranges above does not include the histidine tag residues. Thus, in one aspect, histidine residues at either end of the peptide are discounted when determining the numbering of amino acids in the modified peptide. In another aspect, all amino acid residues are counted including those making up a histidine tag.

In a preferred aspect of the invention the peptide comprises 3 to 20 (preferably contiguous) amino acids, for example 3 to 16 amino acids. Preferably still the peptide comprises 5 to 14 amino acids. In some aspects, the peptide may comprise 12 (preferably contiguous) amino acids.

As known to the skilled person, amino acids can be placed into different classes depending primarily upon the chemical and physical properties of the amino acid side chain. For example, some amino acids are generally considered to be hydrophilic or polar amino acids and others are considered to be hydrophobic or non-polar amino acids. Hydrophobic amino acid may be selected from the group of hydrophobic amino acids consisting of glycine, leucine, phenylalanine, proline, alanine, tryptophan, valine, isoleucine, methionine, tyrosine and threonine; cationic amino acids may be selected from the group consisting of ornithine, histidine, arginine and lysine. As used herein, the terms “hydrophobic” and “cationic” may refer to amino acids having a hydrophobicity that is greater than or equal to −1.10 and/or a net charge that is greater than or equal to 0 as described in Fauchere and Pliska Eur. J. Med Chem. 10:39 (1983). A hydrophobic or non-polar amino acid may also refer to an amino acid having a side chain that is uncharged at physiological pH, is not polar and that is generally repelled by aqueous solution. The amino acids may be naturally occurring or synthetic.

Suitably, the arginine residue is the predominant amino acid in the peptide. Suitably, at least 50% of the amino acid residues are arginine residues, preferably at least 60% or at least 70% or at least 80% of the amino acids in the peptide are arginine. Preferably, at least 90% are arginine residues. In some embodiments all the amino acids in the peptide are arginine residues (optionally with the exception of a histidine tag).

Suitably, the peptide may comprise amino acids other than arginine is non-predominant amounts. For example, histidine, ornithine and lysine could be used.

Suitably, 3 to 50 (preferably contiguous) D and/or L amino acids consist of arginine or a combination of arginine and lysine residues except for 0, 1, or 2 substitutions to an amino acid residues other than arginine or lysine. Preferably, such substitutions (if present) are with another cationic amino acids selected from the group consisting of histidine, ornithine and lysine. Preferably the substations are with lysine.

Suitably, the peptide may be substituted with 0, 1, 2, 3, 4, 5, 6, 7 or 8 substitutions provided that the arginine make up at least 60%, preferably at least 75% of the peptide.

Preferably, the amino acids are L-amino acids.

In a preferred aspect of the invention, at least 90%, for example at least 95% such as 97-99% or even 100%, of the amino acids in the peptide are L-amino acids.

The invention also includes known isomers (structural, stereo-, conformational and configurational), peptidomimetics, structural analogues of the above amino acids, and those modified either naturally (e.g. post-translational modification) or chemically, including, but not exclusively, phosphorylation, glycosylation, sulfonylation and/or hydroxylation.

In general, the peptide of the invention does not include the amino acids aspartic acid, glutamic acid, asparagine, glutamine or serine, but certain peptides of the invention may have activity even though these amino acids are present.

One or more of the residues of the peptide can be exchanged for another to alter, enhance or preserve the biological activity of the peptide. Such a variant can have, for example, at least about 10% of the biological activity of the corresponding non-variant peptide. Conservative amino acids are often utilised, i.e. substitutions of amino acids with similar chemical and physical properties as described above. Hence, for example, conservative amino acid substitutions may involve exchanging lysine for arginine, ornithine or histidine; or exchanging arginine for lysine or isoleucine, ornithine for histidine; or exchanging one hydrophobic amino acid for another. After the substitutions are introduced, the variants are screened for biological activity.

### Peptides of the Invention

The term “peptide” as used herein means, in general terms, a plurality of amino acid residues joined together by peptide bonds. It is used interchangeably and means the same as polypeptide and protein.

The term “modified peptide” refers to a peptide comprising 3 to 50 amino acid residues predominantly arginine further comprising: a histidine tag; and/or a fatty acid and/or a pegylated peptide. Suitably, the modified peptides of the present invention, may be linear peptides.

Preferably, the modified peptides of the present invention may consist of:
1) 3 to 50 amino acid residues predominantly arginine and a histidine tag;
2) 3 to 50 amino acid residues predominantly arginine and one or more fatty acids;
3) 3 to 50 amino acid residues predominantly arginine, a histidine tag and one or more fatty acids;
4) a PEGylated peptide of 3 to 50 amino acid residues predominantly arginine and a histidine tag;
5) a PEGylated peptide of 3 to 50 amino acid residues predominantly arginine and one or more fatty acids; or
6) a PEGylated peptide of 3 to 50 amino acid residues predominantly arginine, a histidine tag and one or more fatty acids.

The peptides of the invention may generally be synthetic peptides. The peptides may be isolated, purified peptides or variants thereof, which can be synthesised in vitro, for example, by a solid phase peptide synthetic method, by enzyme catalysed peptide synthesis or with the aid of recombinant DNA technology.

To identify active peptides that have little or no undesired toxicity for mammalian cells, individual peptides, or libraries of peptides, can be made and the individual peptides or peptides from those libraries can be screened for antimicrobial activity and toxicity, including, but not limited to, antifungal, antibacterial, antiviral, antiprotozoal, anti-parasitic activity and toxicity.

The peptides of the invention can exist in different forms, such as free acids, free bases, esters and other prodrugs, salts and tautomers, for example, and the invention includes all variant forms of the compounds.

Thus, the invention encompasses the salt or pro-drug of a peptide or peptide variant of the invention.

The peptide of the invention may be administered in the form of a pharmaceutically acceptable salt. The pharmaceutically acceptable salts of the present invention can be synthesized from the parent peptide which contains a basic or acidic moiety by conventional chemical methods. Generally, such salts can be prepared by reacting the free acid or base forms of the peptide with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The invention thus includes pharmaceutically-acceptable salts of the peptide of the invention wherein the parent compound is modified by making acid or base salts thereof for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Salts of carboxyl groups of a peptide or peptide variant of the invention may be prepared in the usual manner by contacting the peptide with one or more equivalents of a desired base such as, for example, a metallic hydroxide base, e.g. sodium hydroxide; a metal carbonate or bicarbonate such as, for example, sodium carbonate or bicarbonate; or an amine base such as, for example, triethylamine, triethanolamine and the like.

The invention includes prodrugs for the active pharmaceutical species of the described peptide, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular structures which are rapidly transformed in vivo to the parent structure, for example, by hydrolysis in blood.

A further aspect of the invention provides a pharmaceutical composition comprising a pharmaceutically effective amount of a peptide of the invention, or two or more different peptides of the invention.

The composition also includes a pharmaceutically acceptable carrier, excipient or diluent. The phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

The peptide of the invention is useful, inter alia, as an antimicrobial peptide, for example, against bacteria, fungi, yeast, parasites, protozoa and viruses. The term, “antimicrobial peptide” can be used herein to define any peptide that has microbicidal and/or microbistatic activity and encompasses, non-exclusively, any peptide described as having anti-bacterial, anti-fungal, anti-mycotic, anti-parasitic, anti-protozoal, antiviral, anti-infectious, anti-infective and/or germicidal, algicidal, amoebicidal, microbicidal, bacterici(o)dal, fungicidal, parasiticidal, protozoacidal, protozoicidal properties.

In a preferred aspect, the invention provides the use of a peptide according to the invention in the manufacture of a medicament for treating a microbial infection.

By “microbial infection” is meant an infection caused by a bacterium, parasite, protozoa, virus or fungus including yeast. In one aspect “microbial infection” refers to infections by yeasts and moulds. A “pathogen” is generally defined as any disease-causing organism.

A bacterial pathogen may be derived from a bacterial species selected from the group, but not exclusive to the group, consisting of: Staphylococcus spp., e.g. Staphylococcus aureus (e.g. Staphylococcus aureus NCTC 10442), Staphylococcus epidermidis; Chlamydia spp., e.g. Chlamydia trachomatis, Chlamydia pneumoniae, Chlamydia psittaci; Enterococcus spp., e.g. Enterococcus faecalis; Streptococcus pyogenes; Listeria spp.; Pseudomonas spp.; Mycobacterium spp., e.g. Mycobacterium tuberculosis; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori; Neisseria spp., e.g. Neisseria gonorrhea, Neisseria meningitidis; Borrelia burgdorferi; Shigella spp., e.g. Shigella flexneri; Escherichia coli (Echerichia coli O157:H7 NCTC 12900); Haemophilus spp e.g. Haemophilus influenzae; Francisella tularensis; Bacillus spp., e.g. Bacillus anthraces; Clostridia spp., e.g. Clostridium botulinum; Yersinia spp., e.g. Yersinia pestis; Treponema spp.; Burkholderia spp.; e.g. Burkholderia cepacia, Burkholderia mallei and Burkholderia pseudomallei.

In a preferred use according to the invention the bacterial pathogen is Staphyloccus aureus or Escherichia coli.

A viral pathogen may be derived from a virus selected from, but not limited to, the group consisting of: Human Immunodeficiency Virus (HIV1 & 2); Human T Cell Leukaemia Virus (HTLV 1 & 2); Ebola virus; human papilloma virus (e.g. HPV-2, HPV-5, HPV-8 HPV-16, HPV-18, HPV-31, HPV-33, HPV-52, HPV-54 and HPV-56); papovavirus; rhinovirus; poliovirus; herpesvirus; adenovirus; Epstein Barr virus; influenza virus, hepatitis B and C viruses, Variola virus, rotavirus or SARS coronavirus.

A parasitic pathogen may be derived from a parasite selected from, but not limited to, the group consisting of Trypanosoma spp. (Trypanosoma cruzi, Trypansosoma brucei), Leishmania spp., Giardia spp., Trichomonas spp., Entamoeba spp., Naegleria spp., Acanthamoeba spp., Schistosoma spp., Plasmodium spp., Crytosporidium spp., Isospora spp., Balantidium spp., Loa, Ascaris lumbricoides, Dirofilaria immitis, Toxoplasma ssp., e.g. Toxoplasma gondii.

In a preferred use according to the invention the microbial infection is a fungal infection.

A fungal pathogen may be derived from a fungus (including yeast) selected from, but not limited to, the genera Candida spp., (e.g. C. albicans), Epidermophyton spp., Exophiala spp., Microsporum spp., Trichophyton spp., (e.g T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp. (e.g Malassezia furfur), Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

In a preferred use according to the invention the fungal pathogen is of the genera Candida spp. or Aspergillus spp. For example the fungal pathogen may be Candida albicans, Aspergillus fumigatus, Aspergillus flavus or Aspergillus niger.

The fungal infection may be a systemic, topical, subcutaneous, cutaneous or mucosal infection. Preferably, the fungal infection may be a systemic or mucosal infection.

The peptides of the invention are potent antimicrobial peptides for a wide variety of pathogenic yeast and moulds. However, the peptides of the invention may also be useful in the treatment of other conditions including, but not limited to, conditions associated with mucosal infections, for example, cystic fibrosis, gastrointestinal, urogenital, urinary (e.g kidney infection or cystitis) or respiratory infections.

The term “treatment” relates to the effects of the peptides described herein that in imparting a benefit to patients afflicted with an (infectious) disease, including an improvement in the condition of the patient or delay in disease progression.

In a further aspect, the invention provides a method of treating or preventing a microbial infection in a subject comprising administering to said subject a therapeutically effective amount of a peptide according to the invention.

In a preferred method of the invention, the microbial infection is a fungal infection. In the method of the invention the peptide is preferably administered orally.

Mammals, birds and other animals may be treated by the peptides, compositions or methods described herein. Such mammals and birds include humans, dogs, cats and livestock, such as horses, cattle, sheep, goats, chickens and turkeys and the like. Moreover, plants may also be treated by the peptides, compositions or methods of the invention.

Where the subject is an animal, the method of the invention may be applied to nail-like features, including, but not exclusive to, hooves, claws and trotters.

To achieve the desired effect(s), the peptide, a variant thereof or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight or at least about 1 mg/kg to about 20 mg/kg of body weight, although other dosages may provide beneficial results. The amount administered will vary depending on various factors including, but not limited to, the peptide chosen and its clinical effects, the disease, the weight, the physical condition, the health, the age of the mammal, whether prevention or treatment is to be achieved, and if the peptide is chemically modified.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the peptides of the invention may be essentially continuous over a pre-selected period of time or may be in a series of spaced doses. Both local and systemic administration is contemplated.

To prepare the composition, peptides are synthesized or otherwise obtained, purified as necessary or desired, and then lyophilized and stabilized. The peptide can then be adjusted to the appropriate concentration and optionally combined with other agents. The absolute weight of a given peptide included in a unit dose can vary widely. For example, about 0.01 mg to about 2 g or about 0.01 mg to about 500 mg, of at least one peptide of the invention, or a plurality of peptides specific for a particular cell type can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Thus, one or more suitable unit dosage forms comprising the therapeutic peptides of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic peptides may also be formulated in a lipid formulation or for sustained release (for example, using microencapsulation, see WO 94/07529, and U.S. Pat. No. 4,962,091). The formulations may, where appropriate, be conveniently presented in discrete unit dosage forms and may be prepared by any of the methods well-known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic peptides of the invention are prepared for oral administration, they are generally combined with a pharmaceutically acceptable carrier, diluent or excipient to form a pharmaceutical formulation, or unit dosage form. For oral administration, the peptides may be present as a powder, a granular formation, a solution, a suspension, an emulsion or in a natural or synthetic polymer or resin for ingestion of the active ingredients from a chewing gum. The active peptides may also be presented as a bolus, electuary or paste. Orally administered therapeutic peptides of the invention can also be formulated for sustained release, e.g., the peptides can be coated, micro-encapsulated, or otherwise placed within a sustained delivery device. The total active ingredients in such formulations comprise from 0.1% to 99.9% by weight of the formulation.

Pharmaceutical formulations containing the therapeutic peptides of the invention can be prepared by procedures known in the art using well-known and readily available ingredients. For example, the peptide can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatine, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the peptides of the invention can include buffering agents such as calcium carbonate, magnesium oxide and magnesium carbonate. Suitable buffering agents may also include acetic acid in a salt, citric acid in a salt, boric acid in a salt and phosphoric acid in a salt. Caplets and tablets can also include inactive ingredients such as cellulose, pregelatinized starch, silicon dioxide, hydroxyl propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatine capsules containing at least one peptide of the invention can contain inactive ingredients such as gelatine, microcrystalline cellulose, sodium lauryl sulphate, starch, talc, and titanium dioxide, and the like, as well as liquid vehicles such as polyethylene glycols (PEGs) and vegetable oil. Moreover, enteric-coated caplets or tablets containing one or more peptides of the invention are designed to resist disintegration in the stomach and dissolve in the more neutral to alkaline environment of the duodenum.

The therapeutic peptides of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic peptides of the invention can also take the form of an aqueous or anhydrous solution or dispersion, or alternatively the form of an emulsion or suspension or salve.

Thus, the therapeutic peptides may be formulated for parenteral administration (e.g. by injection, for example, bolus injection or continuous infusion) and may be presented in unit dose form in ampules, pre-filled syringes, small volume infusion containers or in multi-dose containers. The active peptides and other ingredients may form suspensions, solutions, or emulsions in oily or aqueous vehicles, and may contain formulatory agents such as suspending, stabilizing and/or dispersing agents. Alternatively, the active peptides and other ingredients may be in powder form, obtained by aseptic isolation of sterile solid or by lyophilization from solution for constitution with a suitable vehicle, e.g. sterile, pyrogen-free water before use.

These formulations can contain pharmaceutically acceptable carriers, vehicles and adjuvants that are well-known in the art. It is possible, for example, to prepare solutions using one or more organic solvent(s) that is/are acceptable from the physiological standpoint, chosen, in addition to water, from solvents such as acetone, acetic acid, ethanol, isopropyl alcohol, dimethyl sulphoxide, glycol ethers such as the products sold under the name “Dowanol”, polyglycols and polyethylene glycols, C₁-C₄ alkyl esters of short-chain acids, ethyl or isopropyl lactate, fatty acid triglycerides such as the products marketed under the name “Miglyol”, isopropyl mytrisate, animal, mineral and vegetable oils and polysiloxanes.

Solvents or diluents comprising the peptides of the invention may include acid solutions, dimethylsulphone, N-(2-mercaptopropionyl) glycine, 2-n-nonyl-1,3-dioxolane and ethyl alcohol. Preferably the solvent/diluent is an acidic solvent, for example, acetic acid, citric acid, boric acid, lactic acid, propionic acid, phosphoric acid, benzoic acid, butyric acid, malic acid, malonic acid, oxalic acid, succinic acid or tartaric acid.

Also contemplated are combination products that include one or more peptides of the present invention and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that the peptides might be combined with topical antifungal agents such as ciclopirox olamine, haloprogin, tolnaftate, undecylenate, topical nysatin, amorolfine, butenafine, naftifine, terbinafine, and other topical agents.

Additionally, the peptides may be formulated as sustained release dosage forms and the like. The formulations can be so constituted that they release the active peptide, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g. stents, catheters, peritoneal dialysis tubing, draining devices and the like.

For topical administration, the active agents may be formulated as is known in the art for direct application to a target area. Forms chiefly conditioned for topical application take the form, for example, of creams, milks, gels, powders, dispersion or microemulsions, lotions thickened to a greater or lesser extent, impregnated pads, ointments or sticks, aerosol formulations (e.g. sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic peptides of the invention can be delivered via patches or bandages for dermal administration. Alternatively, the peptide can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 microns to about 200 microns.

Topical administration may be in the form of a nail coating or lacquer. For example, the antifungal peptides can be formulated in a solution for topical administration that contains ethyl acetate (NF), isopropyl alcohol (USP), and butyl monoester of poly[methylvinyl ether/maleic acid] in isopropyl alcohol.

Pharmaceutical formulations for topical administration may comprise, for example, a physiologically acceptable buffered saline solution containing between about 0.001 mg/ml and about 100 mg/ml, for example between 0.1 mg/ml and 10 mg/ml, of one or more of the peptides of the present invention specific for the indication or disease to be treated.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active peptides can also be delivered via iontophoresis, e.g., as disclosed in U.S. Pat. No. 4,140,122; 4,383,529; or 4,051,842. The percentage by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic peptides in an aqueous or non-aqueous base also comprising one or more dispersing agents, solubilizing agents or suspending agents. Liquid sprays can be pumped, or are conveniently delivered from pressurized packs. Drops can be delivered via a simple eye dropper-capped bottle, via a plastic bottle adapted to deliver liquid contents drop-wise, or via a specially shaped closure.

The therapeutic peptide may further be formulated for topical administration in the mouth or throat. For example, the active ingredients may be formulated as a lozenge further comprising a flavoured base, usually sucrose and acacia or tragacanth; pastilles comprising the composition in an inert base such as gelatine and glycerine or sucrose and acacia; and mouthwashes comprising the composition of the present invention in a suitable liquid carrier. Alternatively, the active ingredients may be formulated as a film strip or buccal tablet, which may or may not be dissolvable.

Specific non-limiting examples of the carriers and/or diluents that are useful in the pharmaceutical formulations of the present invention include water and physiologically acceptable buffered saline solutions such as phosphate buffered saline solutions pH 7.0-8.0.

The peptides of the invention can also be administered to the respiratory tract. For administration by inhalation or insufflation, the composition may take the form of a dry powder, for example, a powder mix of the therapeutic agent and a suitable powder base such as lactose or starch. Therapeutic peptides of the present invention can also be administered in an aqueous solution when administered in an aerosol or inhaled form. Thus, other aerosol pharmaceutical formulations may comprise, for example, a physiologically acceptable buffered saline solution containing between about 0.001 mg/ml and about 100 mg/ml for example between 0.1 and 100 mg/ml, such as 0.5-50 mg/ml, 0.5-20 mg/ml, 0.5-10 mg/ml, 0.5-5 mg/ml or 1-5 mg/ml of one or more of the peptides of the present invention specific for the indication or disease to be treated.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### FIGURES

FIG. 1 shows the Antimicrobial activity of novel AMPs versus C. albicans SC5314. (A) Efficacy of AMPs was evaluated by MIC after 24 h incubation at 37° C. (B) Efficacy of AMPs of same length was evaluated by MIC after 24 incubation at 37° C. Data is expressed as percentage of the positive growth controls. Data is the mean±standard error of the mean (n=3).

FIG. 2 shows the antifungal activity of AMPs in the presence of 10% saliva. AMPs of the same sequence length were tested versus C. albicans SC5314 in the presence of 10% (v/v) filtered human saliva at pH 7.0. Positive control is 1×RPMI-1640+cells, no peptide. Data is expressed as percentage of the positive growth controls. Data is the mean±standard error of the mean. (n=3)

FIG. 3 shows SEM pictures of lipopeptide treated and untreated Aspergillus species. A and B are A. flavus 01-1554, C and D are A. fumigatus 2002/066 and E and F are A. niger 01-1494. A, C and E are untreated while B, D and E are treated with 250 μg/ml (Rx3)-C16.

FIG. 4 shows the time of kill of (Rx14)-C12 and Rx14 against A. fumigatus and against C. albicans. Cells were incubated at 30° C. with relevant peptide in PBS for set time points then inoculated on SAB plates and again incubated at 30° C. until visible colonies could be seen. Percentage of live cells was calculated by comparing to control (PBS and cells alone).

FIG. 5 shows the stability of lipopeptides in rat plasma and how it affects its activity. Peptide concentrations were incubated with equal volume of rat plasma for time points shown then incubated with CLSI standards amount of Aspergillus spores for 48 h. Percentage activity was calculated by comparing to control which contained no rat plasma.

FIG. 6 shows the MIC data in RPMI-1640 after 48 h.

FIG. 7 shows MIC data in RPMI-1640 and 25% plasma after 24 h.

### EXAMPLES

The following Example illustrates the invention.

### Example 1

### Materials and Methods

The antimicrobial peptides used in Example 1 are listed in Table 1. The manufacturers analysed the peptide for identity by mass spectrometry, and purity and acetate content by high-performance liquid chromatography. The peptide purity ranged from 65 to >95.5%.

TABLE 1
Summary of antimicrobial peptides used

| | | | Purity |
|---|---|---|---|
| AMP | Peptide sequence* | Manufacturer | (%) |
| Novamycin ® | RRRRRRRRRRRRRR | Almac Sciences | 88.7 |
| NP337 | RRRRRRRRRRRRR | Almac Sciences | >95 |
| NP339 | dRdRdRdRdRdRdRdRdRdRdRdRdR | Almac Sciences | 96.1 |
| NP375 | Palm-dRdRdRdRdRdRdRdRdRdRdRdR- | Neo Mps - Poly | >95.5 |
| | CONH | peptide | |
| NP376 | dHdHdRdRdRdRdRdRdRdRdRdRdRdRdR- | Neo Mps - Poly | >95.5 |
| | CONH | peptide | |
| NP377 | dRdRdRdRdRdRdRdRdRdRdRdRdRdHdH- | Neo Mps - Poly | 98.1 |
| | CONH | peptide | |
| NP108 | (1-K)n, n = 77-155, HBr salt | Sigma - Fluka | 85.4 |
| NP121 | Poly-L-Arg, HCl salt | Almac Sciences | >95 |
| NP365 | dHdHdHdHdHdHdHdHdHdHdHdHdHdHdH | Almac Sciences | >95.5 |
| NP466 | RRRFRFRFRFRRR | Almac Sciences | >90 |
| NP319 | RRRRRRRRRRRRRRR | Poly peptide | 92.1 |

*R—arginine, I—Isoleucine, l—Lysine, H—histidine, F—phenylalanine, d—Indicates D-isomer of amino acid, hexanoic—hexanoic acid, octanoic—octanoic acid

### Preparation of AMPs for Experimental Testing

Peptide stock solutions were prepared to 10× concentration required for MIC testing in the desired aqueous solution dependent on the experimental method to be undertaken; dH₂O, 2× or 1×RPMI-1640 or 1×HBSS (Hank's Balanced Salt Solution). Preparation was performed under aseptic conditions in a class two safety cabinet. The net mass of the peptide was taken into account (overall mass×purity) when making stock solutions.

### Preparation of Antifungals for MIC Testing

Nystatin was prepared to the desired stock concentration for MIC testing in dH₂O. Antifungals were stored at −20° C. before use for up to 1 month.

### Determination of Minimum Inhibitory Concentration (MIC) in Presence of Sterile Filtered Saliva

1×RPMI-1640 Buffered with Citrate Phosphate Buffer

Citrate phosphate buffer was prepared by mixing 0.1 M citric acid and 0.2 M dibasic sodium phosphate dodecahydrate in the proportions indicated in the Table 2 and the volume adjusted to 90 ml with dH₂O.

TABLE 2
Volume of citric acid and sodium phosphate used to prepare
citrate phosphate buffer pH 3.4-7.

| | | | | | | | | |
|---|---|---|---|---|---|---|---|---|
| 0.1M Citric | 35.9 | 32.3 | 26.7 | 24.3 | 22.2 | 19.7 | 19.6 | 6.5 |
| acid (ml) | | | | | | | | |
| 0.2M Sodium | 14.1 | 17.7 | 23.3 | 25.7 | 37.8 | 30.3 | 36.4 | 43.6 |
| phosphate (ml) | | | | | | | | |
| pH | 3.4 | 3.8 | 4.6 | 5 | 5.4 | 5.8 | 6.6 | 7 |

pH buffered medium was prepared by dissolving 1.04 g RPMI-1640 premix powder in 10 ml citrate phosphate buffer. The volume was then adjusted to 40 ml with dH₂O and the pH adjusted by addition of the acid or base. The volume was adjusted to 50 ml with dH₂O. The medium was then filter sterilized by vacuum filtration through a 0.2 μm filter and stored at 4° C. and kept for up to 2 weeks.

### Results

The oral cavity is a site which provides a number of challenges that must be overcome if AMPs are to be used in the treatment of OPC. Firstly, the oral cavity has a pH between 5.5 and 7 in disease states whereas the normal pH of the healthy oral cavity is maintained around pH 7 when not feeding. pH influences the charge of AMPs (Mason et al, 2006).

NovaBiotics AMPs are cationic in nature and therefore variation in pH will alter the overall charge of the AMPs. The overall effect pH will have on an AMP is a factor of the number of cationic amino acids, positioning of cationic amino acids, and type of amino acid. In addition to the pH change seen in disease states secreted saliva also contains proteases that aid the breakdown of proteins and therefore peptides. It is advantageous for AMPs intended for use in the oral cavity to have a stable activity spectrum over a range of pH values and also have the characteristic to resist the degradative effects of the oral cavity secretions for a sufficient length of time.

The rationale behind these experiments was to evaluate the efficacy of AMPs versus C. albicans SC5314 at neutral pH and without saliva, then to evaluate the effect pH has on the activity spectrum of the peptides and finally briefly evaluate the effect saliva has on the activity of the selected AMPs. For these experiments the comparator AMP during these experiments is NP319 (15R, +14.9 charge at pH 7).

Efficacy of Novel AMPs Versus C. albicans SC5314

The efficacy of AMPs was determined versus C. albicans SC5314 before evaluating the effect of pH on activity. Addition of a 2 histidine tag on the N terminus (NP376, +13.4 charge at pH 7) was chosen. Substitution of arginine for phenylalanine in NP466 to investigate the reduction in charge was also investigated (+8.9 charge at pH 7). The effect of palmitoylation of NP375 (+11.9 charge at pH 7) was also investigated as this will alter the hydrophobicity of the AMP. Two high weight molecular AMPs were investigated; that of NP108, an isoleucine and lysine AMP, and NP121, a poly L-arginine AMP.

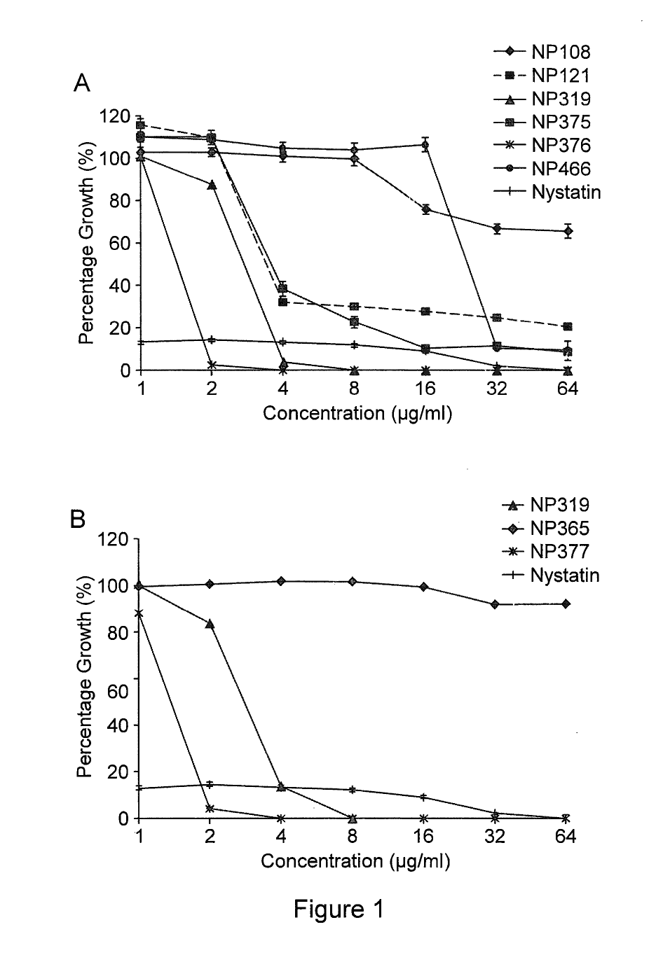

NP376 was the most effective AMP tested in this screen; the MIC₁₀₀ was 2 μg/ml (FIG. 1A). The MIC₁₀₀ of NP341 was 4 μg/ml, the MIC₁₀₀ of NP375 was 16 μg/ml. NP466 did not achieve a MIC₁₀₀, however, an MIC₉₀ of 32 μg/ml was observed. The high molecular weight AMPs did not achieve an MIC₁₀₀ however an MIC₈₀ at 64 μg/ml was seen with NP121 and an MIC₃₀ was seen at 64 μg/ml. Due to a limit in peptide available NP376 could no longer be used and NP377 was substituted in its place for future experiments. At neutral pH alteration of the charge appears to play some role in the efficacy of the AMPs. NP376 was the most effective AMP having a charge of +13.4 and NP319 has a charge of +14.9, this may indicate that within this charge range the variation seen in activity is based on properties other than charge. In comparison NP466 and NP375 were markedly less effective than NP376 and NP319, having a charge of +8.9 and +11.9 respectively, which could be a factor in showing less efficacy versus C. albicans SC5314.

Based on these findings, and to determine if the efficacy of NP377 was similar to that of NP376 efficacy versus C. albicans SC5314, MIC testing was performed. In addition, due to the increased efficacy of the histidine tagged AMP, the efficacy of a histidine rich AMP was evaluated at neutral pH. The MIC₁₀₀ of NP377 against C. albicans SC5314 was 2 μg/ml, the MIC₁₀₀ of NP319 and NP377 was 32 μg/ml. NP365, did not inhibit C. albicans SC5314 growth (FIG. 1B).

### The Effect of pH on the Activity of AMPs

The pH of RPMI-1640 was altered, using citrate phosphate buffer, to reflect the more acidic pH environment in the oral cavity. NP319 and NP377 were active against C. albicans SC5314 from pH 5.5-7.0 (Table 2). For all AMPs tested the optimum pH for activity was pH 5.5. NP377 maintained activity at 2 μg/ml from pH 5.5-7 with reduced activity at pH 5. NP365 had some activity at pH 5.5-6.0 but inhibition of growth by MFC testing was determined to be fungistatic and not fungicidal, unlike the other AMPs.

These results demonstrate that NP319 & NP377 retain antifungal activity in the presence of filtered human saliva. No loss of activity for NP377 and NP319 was seen between pH 5.5-pH 6.5. Below pH 5 the activity of all peptides tested was eliminated against C. albicans SC5134. Alteration of charge was calculated using the online tool Protein calculator v3.3 (Scripss edu, 2006). NP319 shows little charge variation already being highly cationic: pH 7=+14.9, pH 6.5-pH 5=+15. NP377 shows greater charge variation: pH 7=+13.4, pH 6.5=+14, pH 6=+14.5, pH 5.5=+14.8 and pH 5=+15.

TABLE 2
The effect of pH on the antifungal activity of AMPs against C. albicans
SC5314. Units are μg/ml. (n = 3)

| | pH Summary | | | | | | | | | | | |
|---|---|---|---|---|---|---|---|---|---|---|---|---|
| | 4.5 | | 5 | | 5.5 | | 6 | | 6.5 | | 7 | |
| | MIC₁₀₀ | MIC₅₀ | MIC₁₀₀ | MIC₅₀ | MIC₁₀₀ | MIC₅₀ | MIC₁₀₀ | MIC₅₀ | MIC₁₀₀ | MIC₅₀ | MIC₁₀₀ | MIC₅₀ |
| NP319 | >64 | 64 | 32 | 8 | 4 | 2 | 4 | 2 | 2 | 2 | 8 | 4 |
| NP365 | >64 | >64 | >64 | 64 | 64 | 8 | >64 | 32 | >64 | 32 | >64 | >64 |
| NP377 | >64 | 32 | 8 | 4 | 2 | 1 | 2 | 2 | 2 | 2 | 4 | 2 |
| Nystatin | 2 | 2 | 1 | 1 | 1 | 1 | 1 | 1 | 1 | 1 | 1 | 1 |

NP365 shows the greatest increase in charge that directly results in increased efficacy versus C. albicans SC5314. NP365: pH 7=+3.5, pH 6.5=+7.5, pH 6=+11.4, pH 5.5=+13.6 and pH 5=+14.6. The difference in efficacy of NP377 and NP319 is due to the double histidine tag on the C terminus of NP377, however, it is not obvious as to why this is, as it is not a factor of charge alone (both are highly cationic).

Screen of AMPs Versus C. albicans SC5314 in the Presence of Saliva

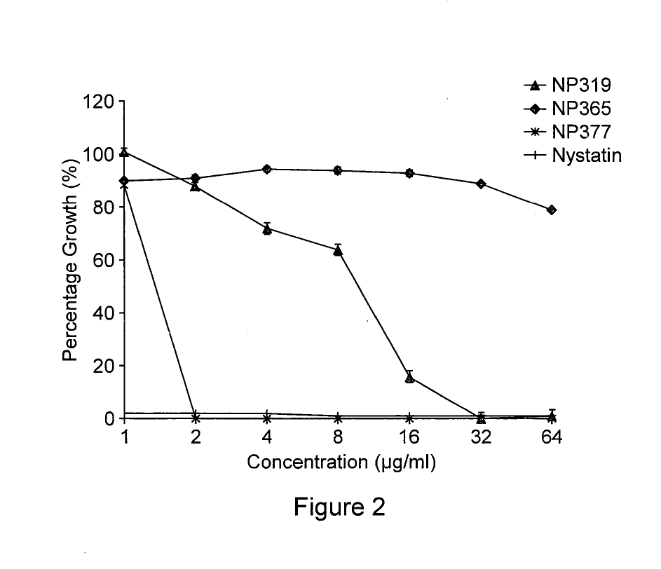

To test if saliva protease could result in a reduction in AMP antifungal activity against C. albicans, antifungal susceptibility testing was conducted in the presence of 10% (v/v) sterile filtered human saliva. NP377 proved to be the most active versus C. albicans SC5314 in 10% saliva with an MIC₁₀₀ of 2 μg/ml (FIG. 2). NP319 had an MIC₁₀₀ of 32 μg/ml and a MIC₅₀ of 8-16 μg/ml and NP377 had an MIC100 at 2 μg/ml.

At all concentrations tested NP365 failed to demonstrate a MIC₅₀. NP377 has a 2 histidine substitution on the C-terminus, otherwise it is identical to NP319. NP365 has the same number of amino acids as NP377 and NP319 but is solely comprised of histidine which is used for the amino acid tag in NP377. Histidine may advantageously provide some protective measure to the proteolytic activity of salivary proteases which would in part explain why no reduction in activity of NP365 and NP377 was observed.

These data show that NP377 has an advantage over the other AMPs due to a tolerance over acid pH range and maintained activity in saliva.

### DISCUSSION

Modification of N and C-terminus with histidine amino acids resulted in an increased stability for the AMP NP377 (R₁₃H₂) over NP319 (R₁₅) in saliva and also demonstrated a better efficacy over the pH range 5-7.

The optimum pH for efficacy of NP319 & NP377 was found to be between pH 5.5-6.5. Due to the increased efficacy demonstrated at pH 5.5-6.5, a formulation strategy that combines a pH stabilising agent with AMPs would be an attractive option for oral formulations.

### Example 2

### Materials and Methods

### Materials

All chemicals used were purchased from Sigma-Aldrich, unless otherwise stated. All peptides were synthesised by PolyPeptide Laboratories, Strasbourg, France SAS.

### Fungal Strains and Mammalian Cell Lines and Growth Conditions

The fungal strains A. fumigatus (AM2002/066 and NPCF2939), A. flavus (01-1554 and NPCF7117) and A. niger (01-1494 and NCPF2022) were obtained from the National Collection of Pathogenic Fungi (NPCF) or American Type Culture Collection (ATCC). Fungal strains were grown on SAB slopes for 72 h at 30° C. before experimental use. C. albicans strain SC5314 was purchased from ATCC and grown overnight on SAB slopes at 30° C. before experimental use.

The cell lines A549 (Human Lung Adenocarcinoma Epithelial Cell Line) and HepG2 (Human Hepatocellular Carcinoma Cell Line) were purchased from ATCC. A549 cells were maintained in F-12K medium (Gibco) containing 10% foetal bovine serum (Life Technologies) and 1% penicillin/streptomycin (Life Technologies) and HepG2 cells were maintained in Dulbecco's modified eagles medium (Gibco) containing 10% foetal bovine serum (Life Technologies), penicillin/streptomycin (Life Technologies) and 1% 0.01 mg/ml human transferrin (Sigma). Both were incubated at 37° C. with 5% CO₂ until cells reached >80% confluence before splitting or experimental use.

### Antifungal Activity and MFC

The antifungal activity of peptides was examined using the conditions of the Clinical Laboratory Standards Institute document M38-A2. Peptide antifungal activity was assessed by micro-broth dilutions with a serial two-fold dilution of test compound prepared in a 96 well plate from 0 to maximum concentration to be tested (here 2× concentration required, 250 μg/ml). Fungal suspensions were prepared by flooding slopes with PBS and filtering through gauze to collect spores. The suspension was then adjusted to a density of 2×10⁴ colony-forming units/ml in 2× culture medium (RPMI 1640, 0.165 M MOPS, pH 7.4 with L-glutamine, without NaHCO₃ medium) and an equal volume was added to the different peptide concentrations in the 96 well plate. Plates where incubated at 30° C. for 48 h before growth inhibition was determined by measuring absorbance at 530 nm (Biotek Powerwave). Antifungal activity was defined by the minimum inhibitory concentration (MIC), the concentration at which no growth was observed.

For examining the minimum fungicidal concentration (MFC) 10 μl of fungal and peptide suspension was transferred from every well to the corresponding well in a sterile 96-well plate containing fresh culture medium (RPMI-1640, 0.165 M MOPS, pH 7.4 with L-glutamine, without NaHCO₃ medium). Aspergillus was then incubated for a further 48 h after which absorbance was measured at 530 nm. MFC was defined as the concentration at which no growth was observed.

### Cytotoxicity

Cell lines were split and viability assessed by tryptan blue staining before plating in unsupplemented 1×RPMI 1640 at a density of 4×10⁵ cells/ml in 96-well plates. Before exposure to peptides, the plate was incubated at 37° C. with 5% CO₂ overnight to allow cells adhere. Peptides were prepared in 96 well plates by the micro-broth dilution method as described above with a maximum concentration of 2500 μg/ml with 1×RPMI 1640. Medium was removed from the plate containing cells and peptide dilutions were transferred to cells and incubated for a further 3 h at 37° C. with 5% CO₂. Cytotoxicity was determined by LIVE/DEAD® Viability/Cytotoxicity kit (Invitrogen Life Technologies), a working solution of 2 μM calcein AM and 4 μM EthD-1 and in conjunction Alamar Blue at a final concentration of 10% was added to the wells. Cells without peptides were the negative control and 1% DMSO was the positive control. Cells were incubated for 3 h again at 37° C. with 5% CO₂. Fluorescence was measured at 528 nm excitation and 590 nm emission for Alamar blue, 485 nm excitation and 528 nm emission for Calcein (live) and 528 nm excitation and 645 emission for EthD-1 (dead).

Haemolysis of Human Red Blood Cells (hRBCs)

Peptides were tested for their haemolytic activity against human red blood cells (hRBC). Fresh hRBCs collected in BD Vacutainer® tubes with EDTA were rinsed 3 times with PBS (pH 7.4) by centrifugation for 10 min at 800 g and resuspended, 1 in 10 diluted with PBS. Peptides were prepared in 1× Hank's Balanced Salt Solution (HBSS) by the micro-broth dilution method as described above in a v-bottomed 96-well plate at a maximum concentration of 2500 μg/ml and an equal volume of hRBC suspension added. hRBCs were then incubated at 37° C. with 5% CO₂ for 3 h. Release of haemoglobin was then calculated by transferring supernatant to a flat bottomed 96-well plate and measuring absorbance at 578 nm. hRBCs with no peptides added and H₂O provided the controls.

### In Vitro Stability Testing

An equal volume of rat plasma was added to three peptide concentrations (100, 50 and 25 μg/ml) in a 96 well plate for time periods of 24, 12, 6, 3, 1 and 0 h and incubated at 30° C. At zero time point A. fumigatus spores were adjusted to a density of 2×10⁴ colony-forming units/ml as described above and added in equal volume to the rat plasma with varying peptide concentrations to give final concentrations of peptide of 50, 25 and 12.5 μg/ml. Controls included peptide concentrations containing no rat plasma and inoculated and uninoculated controls. Plates were incubated at 30° C. for 48 h after which absorbance was measured at 530 nm. Peptide plasma stability was defined as a visible increase in MIC value.

### Time of Kill

Mature A. fumigatus 2002/066 spores were isolated as described above and mature C. albicans SC5314 cells scraped from slopes were adjusted to 2,000 cells/ml in PBS. For A. fumigatus 2002/066, the fungal suspension was incubated in a 96 well plate with an equal volume of peptide (Rx14)-C12 at 8 and 16 μg/ml. For C. albicans SC5314, the fungal suspension was incubated in a 96 well plate with an equal volume of peptide (Rx14)-C12 and peptide Rx14 at 8 and 16 μg/ml. Plates were incubated at 30° C. for periods of 0, 0.5, 1, 2, 4, 6, 24 and 30 h after a 1:10 dilution was made and plated in triplicate onto SAB plates and further incubated for 48 h or until visible colonies could be identified. Controls included cells plus PBS. Time of kill was defined as the number of colonies reduced by >90% compared to positive control.

### Scanning Electron Microscopy

2×10⁴ mature A. fumigatus 2002/066, A. flavus 01-1554 and A. niger 01-1494 spores were grown in RPMI 1640 in a 24 welled plate containing a 0.2 μm pore size Whatman® Polycarbonate filter for 48 h. Medium supernatant was removed and 1 ml of RPMI-1640 containing 250 μg/ml of peptide was added and incubated for 1 h at 30° C. The medium was again removed and fixed in 2.5% glutaraldehyde in 0.1 M phosphate buffer pH 7.2-7.4 for 24-48 hours. The samples were then washed with 0.1M phosphate buffer 4×15 min then rinsed with distilled water 3×5 min. Finally, the samples were dehydrated in ethanol before being dried then coated in a vacuum with an electrically conductive layer of gold. Images were then viewed using a Zeiss EVO MA10 Scanning Electron Microscope.

### Results

### Antifungal Activity of Proprietary NovaBiotics Lipopeptides

A set of novel lipopeptides were assayed against the major pathogenic Aspergillus species (A. fumigatus, A. flavus and A. niger) according to recorded aspergillosis case reports (Summerbell, R. (2003) Ascomycetes—Aspergillus, Fusarium, Sporothrix, Piedraia, and their relatives. Pathogenic fungi in humans and animals. Marcel Dekker Inc.). The peptides contained different numbers of arginine residues with a sixteen carbon ring (C16) attached at either end. The antifungal drugs amphotericin B, itraconazole and caspofungin served as controls. The data shown in Table 3 indicates that three amino acids attached to the terminal of the sixteen carbon lipid provided the best overall activity of all the lipopeptides tested (2 μg/ml for A. niger and 4 μg/ml for A. fumigatus and A. flavus). Increasing the number of amino acids above three increased the MIC values. The terminal location of the lipid impacted on activity. Lipopeptides C16-(Rx12) and (Rx11)-C16 are of similar composition, yet (Rx11)-C16 has smaller MIC values for A. fumigatus and A. niger but has greater MIC values for A. flavus. Although good activity was seen with these lipopeptides. Interestingly, a reduction in haemolysis was observed when the lipid was transferred to the centre of the peptide sequence. For example, the lipopeptide (Rx6)-C16-(Rx6) had a reduced haemolytic activity of 26% compared to the lipopeptide (Rx11)-C16 which is of similar composition but had a haemolytic value of 98% at 2.5 mg/ml.

A range of lipid carbon chain lengths (at 200 μg/ml) were then tested for their effects on A. fumigatus growth, and cytotoxicity in order to separate any effects solely due to the lipids versus entire the lipopeptide structure. The data in Table 4 show that there was a bell-shaped effect between carbon length of the lipid and A. fumigatus growth inhibition. Medium carbon chain lengths had the greatest activity (C6, C8, C10 and C12 inhibited growth by 50, 60, 20 and 45% respectively compared to C2, C4, C14 and C16 which inhibited growth by 5, 35, 15 and 0% respectively). At 200 μg/ml there was a slight decrease in haemolysis as the carbon chain increased (40, 30 and 40% for C2, C4 and C6 compared to 20, 20 and 15% for C12, C14 C16 respectively).

Following on from this it was determined that increasing the number of arginine amino acids attached to a twelve carbon lipid provided higher anti-aspergillus activity (Table 5), although an MIC of >250 μg/ml was seen for A. flavus for example (Rx14)-C12. Insertion of lipids at the terminus provided the optimal MIC values. (Rx14)-C12 had an MIC of 4 μg/ml for A. fumigatus and 16 μg/ml for A. niger but a central location of the lipid increased the MIC value 1 to 2 fold, (Rx7)-C12-(Rx7) had an MIC of 16 μg/ml for A. fumigatus and 32 μg/ml for A. niger. All twelve carbon lipopeptides tested had no haemolytic activity and no LD₅₀ against A549 cells at 2.5 mg/ml. Rx14 without the lipid moiety had no activity against any of the Aspergillus species tested but had activity against C. albicans. Conversely (Rx14)-C12 and Rx14 had the same MIC value against C. albicans of 4 μg/ml. The lipopeptides (Rx14)-C12 and (Rx7)-C12-(Rx7) had a minimum fungicidal concentration (MFC) that was equal to their minimum inhibitory concentrations (MIC₁₀₀).

### Scanning Electron Microscopy of Peptide-Treated Aspergillus Species

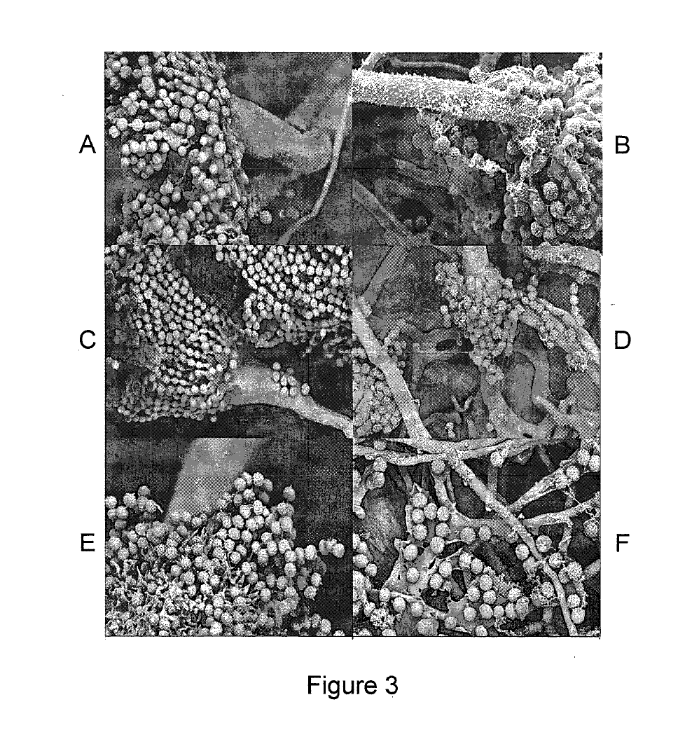

The morphology of (Rx3)-C12 peptide-treated Aspergillus biofilms was examined using SEM. A. fumigatus, A. flavus and A. niger biofilms were each grown under the same conditions and treated with 250 μg/ml (Rx3)-C12 for 1 hour. All biofilms displayed irregular surfaces compared to the untreated control (FIG. 3).

TABLE 3
Antifungal MICs and cytotoxicity of hit lipopeptides (μg/ml).

| | Aspergillus MIC | | | Cytotoxicity | Haemolysis |
|---|---|---|---|---|---|
| | A. fumigatus | A. niger | A. flavus | A549 cells | % RBCs lysed |
| Peptides | MIC₁₀₀ | MIC₁₀₀ | MIC₁₀₀ | (LD₅₀) | at 2.5 mg/ml |
| (Rx3)-C16 | 4 | 2 | 4 | 20 | 100 |
| (Rx5)-C16 | 8 | 6 | 32 | 30 | 100 |
| (Rx11)-C16 | 6 | 6 | 64 | 30 | 98 |
| (Rx3)-C16-(Rx3) | 16 | 2 | 32 | 24 | 88 |
| (Rx5)-C16-(Rx5) | 16 | 2 | 64 | 30 | 60 |
| (Rx6)-C16-(Rx6) | 32 | 4 | 64 | 30 | 26 |
| C16-(Rx12) | 16 | 6 | 32 | 30 | 87 |

MIC values shown are averages taken from two strains of each species, A. fumigatus (AM2002/066 and NPCF2939), A. flavus (01-1554 and NPCF7117) and A. niger (01-1494 and NCPF2022).

TABLE 4
Effect of lipids on A. fumigatus, A549 cells and RBCs

| | % Growth | % A549 | % Haemolytic |
|---|---|---|---|
| Systematic name and | Inhibition of | Cell Death | Activity |
| Carbon length | A. fumigatus at 48 h | at 3 h | at 3 h |
| Acetic (C2) | 5 | 30 | 40 |
| Butanoic (C4) | 35 | 0 | 30 |
| Hexanoic (C6) | 50 | 0 | 40 |
| Octanoic (C8) | 60 | 5 | 35 |
| Decanoic (C10) | 20 | 25 | 10 |
| Dodecanoic (C12) | 45 | 20 | 20 |
| Tetradecanoic (C14) | 15 | 0 | 20 |
| Palmitic (C16) | 0 | 10 | 15 |

TABLE 5
Antifungal MICs and MFCs of target lipopeptides (μg/ml).

| | Aspergillus^{#} | | | | | | Candida | | Cytotoxic | Haemolysis |
|---|---|---|---|---|---|---|---|---|---|---|
| | A. fumigatus | | A. niger | | A. flavus | | C. albicans | | A549 | RBCs |
| | MIC₁₀₀ | MFC | MIC₁₀₀ | MFC | MIC₁₀₀ | MFC | MIC₁₀₀ | MFC | (LD50) | % at 2500 |
| (Rx3)-C12 | n/a | | n/a | | n/a | | | | >2500 | 0 |
| (Rx10)-C12 | 8 | | 125 | | n/a | | | | >2500 | 0 |
| (Rx14)-C12 | 4 | 4 | 16 | 16 | >250 | | 4 | | >2500 | 0 |
| (Rx3)-C12-(Rx3) | 64 | | 125 | | n/a | | | | >2500 | 0 |
| (Rx5)-C12-(Rx5) | n/a | | n/a | | n/a | | | | >2500 | 0 |
| (Rx7)-C12-(Rx7) | 16 | 16 | 32 | 32 | >250 | | 8 | | >2500 | 0 |
| Rx14 | n/a | | n/a | | n/a | | 4 | | >2500 | 0 |
| Amphotericin B | 1 | | <1 | | 1.5 | | | | | |
| Itraconcazole | <1 | | <1 | | 1 | | | | | |
| Caspofungin | 64 | | 16 | | 128 | | | | | |
| Fluconazole | 64 | | 64 | | 64 | | | | | |

^{#}MIC values shown are averages taken from two strains of each species, A. fumigatus (AM2002/066 and NPCF2939), A. flavus (01-1554 and NPCF7117) and A. niger (01-1494 and NCPF2022) except for C. albicans SC5314 was the only strain tested.
n/a means not active at 250 μ/ml.

### Time of Kill

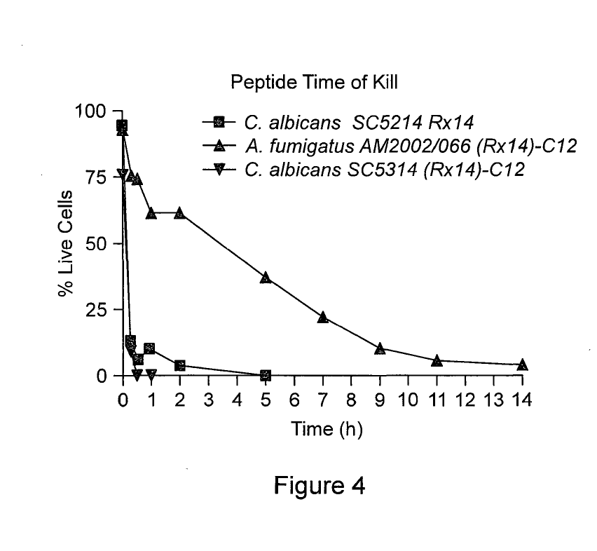

The time of kill of the different lipopeptides was compared as it would indicate any mode of action similarities between them. After diluting the 2,000 spores with an equal volume of peptide and diluting it after the incubation period, the final concentration of spores that was plated was 100 per plate and converted to percentage alive. The time of kill for lipopeptide (Rx14)-C12 against A. fumigatus was 30 h whereas peptides (Rx14)-C12 and Rx14 took 15 min to kill C. albicans (FIG. 4). The considerable difference in the time of kill times between the two fungi would indicate dissimilar mode of action. However, for C. albicans (Rx14)-C12 is suspected to retain the same mode of action as (Rx14) as they have similar time of kills.

### Metabolic Stability

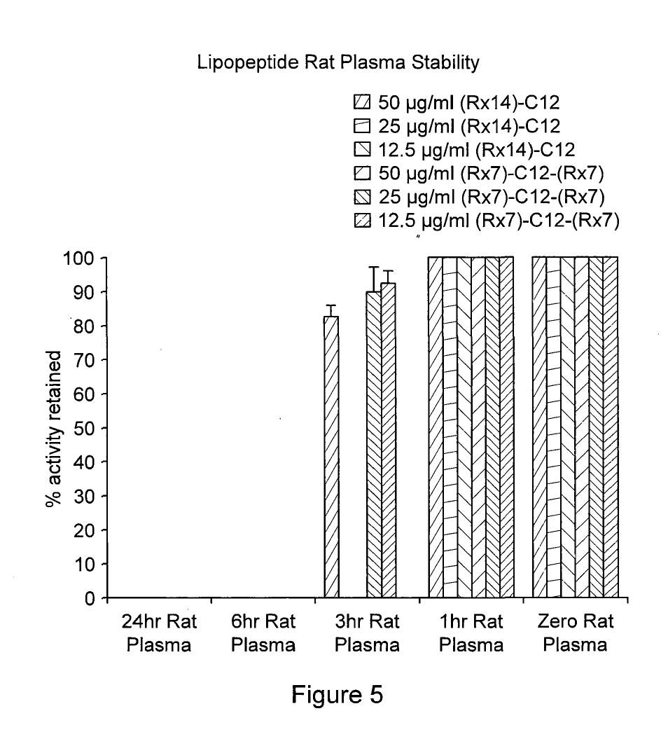

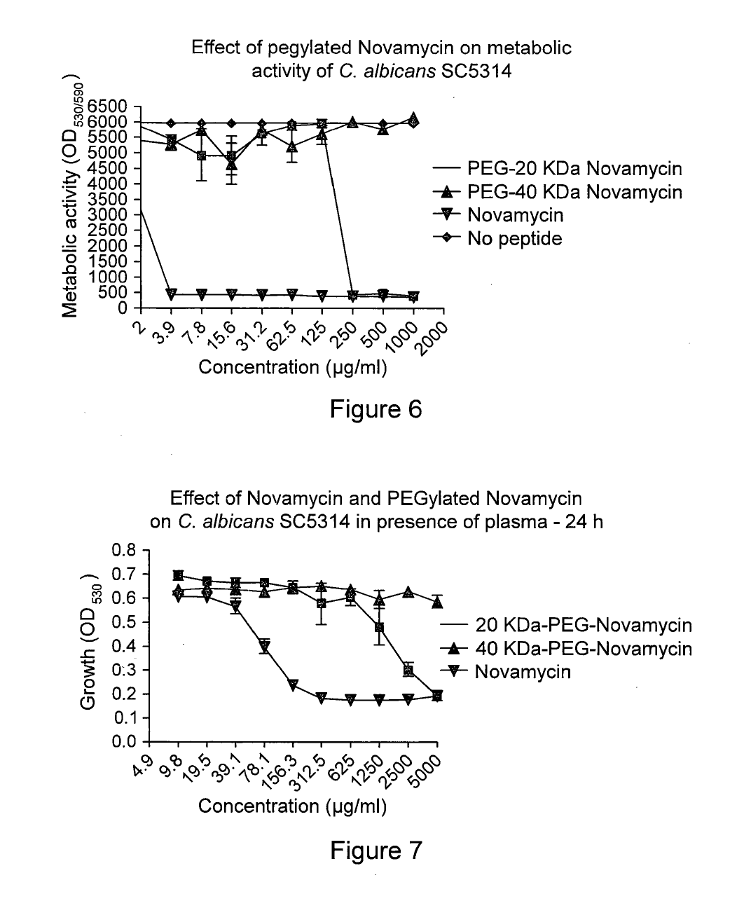

Most human proteins, except antibodies, are rapidly cleared from circulation, typically by renal filtration giving them a half life of only minutes to hours. Stability of (Rx14)-C12 and (Rx7)-C12-(Rx7) was assessed by incubating rat plasma with 50, 25 and 12.5 μg/ml of peptide for 24, 6, 3 and 1 h then comparing their anti-A. fumigatus activity. Both lipopeptides displayed similar plasma half-lives however (Rx14)-C12 had a slightly shorter half-live (FIG. 5). At 3 h, only 87% of 50 μg/ml of (Rx14)-C12 retained its activity whereas at concentrations 25 and 12.5 μg/ml all activity was lost. As for (Rx7)-C12-(Rx7) at 3 h, 95% of 50 μg/ml activity and 90% of 25 μg/ml activity was retained and 12.5 μg/ml lost all activity. All values were compared to the relevant lipopeptide alone control. At 1 hour all activity was retained.

### Discussion

Characteristically most endogenous cationic antimicrobial peptides (AMPs) contain 12-50 amino acids, whereas antimicrobial lipopeptides are known for their shorter amino acid chain, 6 or 7 D- and L-amino acids and predominantly containing a complex cyclic structure with a fatty acid side chain. Both peptide classes having very different modes of action. Interestingly, longer (>12C) compared to shorter (<12C) fatty acid chains produced by microorganisms have been shown to have increased antibacterial and antifungal activity. Here in this study it has been found that the attachment of a lipid chain to a linear chain of arginine residues generates potent antifungal activity against the most prevalent invasive fungal pathogens Aspergillus and Candida. We have shown that as with endogenous AMPs the attachment of a short cationic chain, in this case three arginines, and the attachment of an ideal long fatty acid side chain (palmitic acid C16) possessed the best overall antifungal activity against Aspergillus species (Table 3). As the length of the arginine chain increased so did the MIC values for Aspergillus species, substantially for A. flavus and slightly for A. fumigatus and A. niger. However all these peptides displayed cytotoxicity.

After studying the effects of a panel of lipids on A. fumigatus growth (although this was a static effect, data not shown) and on cytotoxicity (Table 4) it was concluded that lauric acid (C12) would be an ideal lipid to attach to an arginine chain. Linear short, medium and large arginine chains with lauric acid terminal and centre located lipopeptides were then tested for their antifungal activity against Aspergillus species. Unlike the lipopeptides with C16 attached, peptides with the longer arginine chain demonstrated greater antifungal activity than the shorter chained versions. However the most promising C12 peptides had larger MIC values for A. niger and A. flavus where a MIC₁₀₀ value wasn't seen at 250 μg/ml. Noteworthy was that all these C12 lipopeptides have zero toxicity at the maximum concentration that they were tested at 2.5 mg/ml. Moreover these lipopeptides are potentially broad spectrum antifungals as they were active against C. albicans. Note that fourteen arginines alone compared to fourteen arginines with lauric acid had identical antifungal activity against C. albicans and only the lipopeptide has antifungal activity against Aspergillus species, suggesting a distinct mode of action between the two peptides. Like most antimicrobial peptides, cidal activity is seen and this is true for (Rx14)-C12 and (Rx7)-C12-(Rx7) as the minimum fungicidal concentrations were identical to the minimum inhibitory concentrations.

To examine the mode of action of these lipopeptides against Aspergillus species and how their different from the non-lipid variant, we first used scanning electron microscopy to visualize any Aspergillus morphology defects after they were treated with a high concentration of lipopeptide. Interestingly, all Aspergillus species tested showed biological material attached to all external surfaces compared to the untreated controls. This biological material is thought to be intracellular components that were released when the cells were lysed by the lipopeptide.

Next differences in the time of kill between the (Rx14)-C12 and Rx14 peptides and Aspergillus and Candida were examined. Here we find that Rx14 had a time of kill of ˜15 min and interestingly ˜15 min was the time required for (Rx14)-C12 to kill C. albicans. As the MIC concentrations and the time of kill are identical between these peptides for C. albicans, this would lead one to suggest that there is a similar mode of action and that the lipid moiety had no known positive or negative effects on drug activity. However the addition of the lipid moiety makes the peptide active against Aspergillus species. In contrast, there was a much longer time of kill for (Rx14)-C12 against A. fumigatus compared to C. albicans suggesting that there is a different mode of action and that the addition of the lipid is crucial for activity against Aspergillus species. It would be important in the future to test (Rx14)-C12 against a broader range of fungi to determine whether it is truly a broad spectrum antifungal.

Peptides have been negatively perceived as therapeutic candidates primarily because of short half lives as well as toxicity issues. As peptide degradation is typically faster in rat plasma than in human plasma or serum, the stability experiment was carried out using rat serum. Stability was assessed by incubating concentrations of the lipopeptides with 100% rat serum before an aliquot was removed at a given time point and incubated with Aspergillus. The results revealed that at one hour all antifungal activity was retained but at three hours (Rx14)-C12 and (Rx7)-C12-(Rx7) lost some antifungal activity. (Rx7)-C12-(Rx7) lost all antifungal activity at 12.5 μg/ml and 25 μg/ml, ˜1 and 2 times respectively their MIC values and retained 85% of 50 μg/ml antifungal activity. However (Rx14)-C12 lost all antifungal activity at 12.5 μg/ml, ˜3 times MIC value but retained its activity at 25 and 50 μg/ml. Although their serum stability seems quite low it is actually very good for a peptide. Moreover, human plasma half life is known to be 3 times that of a rodent and 100% serum doesn't mimic in vivo as whole blood is known to contain ˜55% serum.

To conclude, the data presented in this study indicates that these lipopeptides could be used for aspergillosis therapy and other yeast and mould infections. They demonstrate a promising drug profile having a broad therapeutic window, good antifungal activity and low cytotoxicity.

### Example 3

### Materials and Methods

Materials and Candida albicans Strain Used

The antimicrobial peptides used in Example 3 are Novamycin (as described in Table 1) and Novamycin pegylated with either a 20 kDa PEG (Novamycin-PEG20 KDa) or a 40 kDa PEG (Novamycin-PEG40 KDa) synthesised by AmbioPharm.

All chemicals were purchased from Sigma-Aldrich, unless stated otherwise. The C. albicans culture used was SC5314.

### Preparation of AMPs for Experimental Testing

Novamycin was prepared at a concentration of 20 mg/ml in sterile distilled water and the Novamycin with 20 kDa PEG (Novamycin-PEG20 KDa) and 40 kDa PEG (Novamycin-PEG40 KDa) were prepared at 2 mg/ml for the CLSI based MIC and 20 mg/ml for the MIC in the presence of human plasma. The purity of the peptide was taken into account during the preparation to ensure preparation of the correct concentration. Peptides solutions were stored at −20° C. until required.

### Determination of Minimum Inhibitory Concentration (MIC)

The antifungal activity of the peptides was based on that of the Clinical Laboratory Standards Institute document M27-A3. A serial two-fold dilution of peptide was prepared across a 96-well microtitre plate with concentration ranging from 2 mg/ml to 15.625 mg/ml; upon addition of the cells these concentrations shall be halved, with additional wells lacking peptide as a 0 mg/ml control. An addition control lacking culture was also prepared.

To prepare the fungal inoculum, an overnight culture of C. albicans SC5314 was grown at 30° C. on Sabouraud dextrose agar. Cells were washed from the surface of the agar and diluted to the equivalent of the 0.5 MacFarland standard. A further 1:50 dilution of the cells into 2×RPMI-1640 was carried out, and an equal volume was added to the different peptide concentrations in the microtitre plate. To measure the metabolic activity of the cells, 10 μl of a 0.25% solution of resazurin was added to each well. Plates were incubated in the dark at 30° C. for 48 h with growth inhibition determined by measuring absorbance at 530 nm/590 nm at 0 h, 24 h and 48 h on a BioTek PoweWave. Antifungal activity was defined by the minimum inhibitory concentration (MIC), the concentration at which no growth was observed.

### Determination of Minimum Inhibitory Concentration (MIC) in the Presence of Plasma

To determine the effect of Novamycin and pegylated Novamycin in the presence of human plasma a minimum inhibitory concentration in the presence of 25% human plasma was carried out.

A peptide “master” plate was prepared by the addition of 200 μl of the 20 mg/ml stock to column 11 on a microtitre plate. One hundred microliters of sterile water was added to the remaining wells. Serial two-fold dilutions of the peptides were carried out across the plate from right to left. From the “master” plate, 25 μl of peptides were added to corresponding wells on three “working” plates. To each well on the “working” plate, 25 μl human plasma was added, resulting in the halving of both the concentration of the peptide in the working plate and human plasma. Column 12 contained the controls.

An overnight culture of C. albicans SC5314 was grown at 30° C. on Sabouraud dextrose agar and the cells were washed from the surface of the agar and diluted to the equivalent of the 0.5 MacFarland standard. A further 1:50 dilution of the cells into 2×RPMI-1640 was carried out, and 50 μl of the cell suspension was added to each well of the plate. This resulted in the peptide concentration halving again, to a quarter of the original concentration, and a final concentration of 25% human plasma. Plates were incubated at 30° C. for 48 h with growth inhibition determined by measuring absorbance at 530 nm at 0 h, 24 h and 48 h on a BioTek PoweWave. Antifungal activity was defined by the minimum inhibitory concentration (MIC), the concentration at which no growth was observed.

### Results

TABLE 6
MIC data in RPMI-1640 (48 h data)

| | MIC₅₀ | MIC₈₀ | MIC₁₀₀ |
|---|---|---|---|
| | (μg/ml) | (μg/ml) | (μg/ml) |
| Novamycin | <2 | 2 | 4 |
| Novamycin-PEG20KDa | 125-250 | 250-500 | 250-500 |
| Novamycin-PEG40KDa | >1000 | >1000 | >1000 |

TABLE 7
MIC data in RPMI-1640 and 25% plasma (24 h
data)

| | MIC₅₀ | MIC₈₀ | MIC₁₀₀ |
|---|---|---|---|
| | (μg/ml) | (μg/ml) | (μg/ml) |
| Novamycin | 78.1 | 156.25-312.5 | 312.5 |
| Novamycin-PEG20KDa | 1250-2500 | 2500-5000 | 5000 |
| Novamycin-PEG40KDa | >5000 | >5000 | >5000 |

1. A modified peptide comprising from 3 to 50 D and/or L arginine amino acids except for 0, 1 or 2 substitutions and wherein the peptide further comprises a modification which is selected from one or more of the group consisting of:
1) incorporation of a histidine tag;
2) lipidation; and
3) PEGylation.

2. The modified peptide in accordance with claim 1 wherein the peptide has a histidine tag at the N terminus or C terminus.

3. The modified peptide in accordance with claim 2 wherein the histidine tag has at least two histidine residues.

4. The modified peptide according to claim 1 wherein the modified peptide is a lipidated peptide such that a fatty acid is conjugated to the peptide.

5. The modified peptide in accordance with claim 4 wherein the fatty acid is a C3 to C14 fatty acid.

6. The modified peptide in accordance with claim 5 wherein the fatty acid is a C8 to C14 fatty acid.

7. The modified peptide in accordance with claim 1 wherein the modified peptide is PEGylated.

8. A method of preparing a modified peptide comprising:
1) providing a peptide comprising from 3 to 50 D and/or L arginine amino acids except for 0, 1 or 2 substitutions; and
2) incorporating a histidine tag; conjugating said peptide with a fatty acid and/or PEGylating said peptide, thereby producing the modified peptide.

9. (canceled)

10. A pharmaceutical composition comprising the modified peptide in accordance with claim 1 and a pharmaceutically acceptable carrier, excipient or diluent.

11. The pharmaceutical composition according to claim 10 further comprising a pH stabilising agent.

12. (canceled)

13. The pharmaceutical composition in accordance with claim 10 for use in the prevention or treatment of a microbial infection.

14. The pharmaceutical composition according to claim 13, wherein the microbial infection is a fungal, yeast or mould infection.

15. The pharmaceutical composition according to claim 14, wherein the microbial infection is a Candida spp., Epidermophyton spp., Exophiala spp., Microsporum spp., Trichophyton spp., Tinea spp., Aspergillus spp., Blastomyces spp., Blastoschizomyces spp., Coccidioides spp., Cryptococcus spp., Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp., Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., or Wangiella spp. infection.

16. The pharmaceutical composition according to claim 10 for use in the treatment or prevention of any one or more of the group consisting of: candidiasis, aspergillosis, athlete's foot, basidiodiabolomycosis, blastomycosis coccidioidomycosis cryptoccocis, basal meningitis, dermatophytosis, onchomycosis, dermatophytids, endothrix, exothrix, fungal meningitis, fungemia, histoplasmosis, mycosis, myrinogmycosis, paracoccidioidomycosis, penicilliosis, piedra, pneumocytosis pneumonia, sporptrichosis, tinea, zeospora and zygomycosis.

17. A method of treating or preventing a microbial infection in a subject comprising administering a pharmaceutically effective amount of the modified peptide in accordance with claim 1.

18. The method according to claim 17, wherein the microbial infection is a fungal infection.

19. The method according to claim 18, wherein the fungal infection is a Candida infection and/or an Aspergillus infection.

20. The method according to claim 17 wherein the administration is orally.

21. A method of treating or preventing any one or more of the group consisting of: candidiasis, aspergillosis, athlete's foot; basidiodiabolomycosis; blastomycosis; coccidioidomycosis cryptoccocis; basal meningitis; dermatophytosis; onchomycosis; dermatophytids; endothrix; exothrix; fungal meningitis, fungemia, histoplasmosis, mycosis, myrinogmycosis, paracoccidioidomycosis, penicilliosis, piedra, pneumocytosis pneumonia, sporptrichosis, tinea, zeospora and zygomycosis in a subject, said method comprising administering a pharmaceutically effective amount of the modified peptide in accordance with claim 1.

22. The method according to claim 17 wherein the subject has an immunocomprised state.

23. (canceled)

24. (canceled)

25. (canceled)
